(19) United States
(12) Patent Application Publication
(10) Pub. No.: US 2026/0092257 A1
(43) Pub. Date: Apr. 2, 2026

(54) METHODS AND COMPOSITIONS FOR GENERATING HEMATOPOIETIC PROGENITOR CELLS

(71) Applicant: Trailhead Biosystems Inc., Beachwood, OH (US)

(72) Inventors: Angelica GOMES UELTSCHY, Shaker Heights, OH (US); Michael Elias, Westlake, OH (US)

(21) Appl. No.: 19/347,888

(22) Filed: Oct. 2, 2025

Related U.S. Application Data
(60) Provisional application No. 63/702,502, filed on Oct. 2, 2024.
(60) Provisional application No. 63/855,891, filed on Aug. 1, 2025.

(51) Int. Cl. C12N 5/0789; C12N 5/00

(52) U.S. Cl. CPC C12N 5/0647; C12N 5/0018; C12N 2500/36; C12N 2501/105; C12N 2501/115; C12N 2501/125; C12N 2501/145; C12N 2501/15; C12N 2501/165; C12N 2501/2303; C12N 2501/26; C12N 2506/1353; C12N 2506/1369; C12N 2506/45; C12N 2510/00

(57) ABSTRACT

The invention provides methods for generating human CD34+, CD43+, and CD90+ hematopoietic progenitor cells from hematopoietic stem cells. The methods include the use of chemically-defined culture media. Isolated cell populations are also provided.

### BACKGROUND OF THE INVENTION

Hematopoietic progenitor cells (HPCs) are immature cells that develop from hematopoietic stem cells (HSCs), which support lifelong blood production and are used in therapies for blood disorders. HPCs are defined by the expression of particular markers, including expression of CD34, CD43, and CD90.

While some progress has been made in the field regarding the differentiation protocol for obtaining CD34+, CD43+, and CD90+HPCs from HSCs, there remains a need for efficient methods and compositions for generating HPCs with robust multilineage and lineage-specific potential.

### SUMMARY OF THE INVENTION

This disclosure provides methods of differentiating a population of human hematopoietic stem cells (HSCs), e.g., from cord blood or bone marrow cells, into a population of hematopoietic progenitor cells (HPCs) using chemically-defined culture media that allow for differentiation of CD34+ HSCs in as little as six days of culture. The culture media lack serum and comprises small molecule agents that either agonize or antagonize particular signaling pathway in stem cells such that differentiating along the CD34+, CD43+, and CD90+ HPC lineage is promoted, leading to expression of HPC-associated biomarkers. The methods of the disclosure have the advantage that they significantly shorten the time needed to generate CD34+, CD43+, and CD90+ HPCs. Moreover, the use of small molecule agents in the culture media allows for precise control of the culture components.

Accordingly, in one aspect, the disclosure pertains to a method of generating a population of human CD34+, CD43+, and CD90+ HPCs, the method comprising culturing a population of human CD34+ HSCs in a culture medium comprising a vascular endothelial growth factor receptor (VEGFR) agonist, a fibroblast growth factor receptor (FGFR) agonist, a c-kit agonist, a thrombopoietin receptor (TPOR) agonist, and a fms-like tyrosine kinase 3 (FLT3) agonist for at least 12 hours.

In some embodiments, the VEGFR agonist is selected from the group consisting of vascular endothelial growth factor (VEGF), Gremlin, PG-007, Taurocholic acid, KLTWQELYQLKYKGI, and a peptide derived from VEGF. In some embodiments, the VEGFR agonist is VEGF. In some embodiments, VEGF is present in the culture medium at a concentration of 1-400 ng/ml.

In some embodiments, the FGFR agonist is selected from the group consisting of FGF2, SUN11602, FGF1, FGF3, FGF4, FGF5, FGF6, FGF8, FGF10, FGF17, FGF19, FGF20, FGF21, FGF22, and FGF23. In some embodiments, the FGFR agonist is FGF2. In some embodiments, FGF2 is present in the culture medium at a concentration of 2-150 ng/ml.

In some embodiments, the c-kit agonist is stem cell factor (SCF). In some embodiments, SCF is present in the culture medium at a concentration of 2-200 ng/ml.

In some embodiments, the TPOR agonist is selected from the group consisting of thrombopoietin (TPO), eltrombopag, TA-316, TPO agonist 1, avatrombopag, and lusutrombopag. In some embodiments, the TPOR agonist is TPO. In some embodiments, TPO is present in the culture medium at a concentration of 1-200 ng/ml.

In some embodiments, the FLT3 agonist is selected from the group consisting of FLT3L, CDX-301, GS-3583, FLT3L-Fc, and a peptide derived from FLT3L. In some embodiments, the FLT3 agonist is FLT3L. In some embodiments, FLT3L is present in the culture medium at a concentration of 1-200 ng/ml.

In some embodiments, the method of generating a population of human CD34+, CD43+, and CD90+ HPCs comprises culturing a population of human CD34+ HSCs in a culture medium comprising VEGF, FGF2, SCF, TPO, and FLT3L. In some embodiments, VEGF is present at a concentration of 20 ng/ml, FGF2 is present at a concentration of 10 ng/ml, SCF is present at a concentration of 50 ng/ml, TPO is present at a concentration of 20 ng/ml, and FLT3L is present at a concentration of 20 ng/ml. In another aspect, the disclosure pertains to a method of generating a population of human CD34+, CD43+, and CD90+ HPCs, the method comprising culturing a population of human CD34+ HSCs in a culture medium comprising a VEGFR agonist, an FGFR agonist, a c-kit agonist, an interleukin-3 receptor (IL-3R) agonist, an antioxidant, a FLT3 agonist, a transforming growth factor beta (TGF-β) antagonist, a transient receptor potential vanilloid 4 (TRPV4) agonist, a bioactive phospholipid, and an insulin-like growth factor 1 (IGF-1) receptor agonist for at least 12 hours.

In some embodiments, the VEGFR agonist is selected from the group consisting of VEGF, Gremlin, PG-007, Taurocholic acid, KLTWQELYQLKYKGI, and a peptide derived from VEGF. In some embodiments, the VEGFR agonist is VEGF. In some embodiments, VEGF is present in the culture medium at a concentration of 4-400 ng/ml.

In some embodiments, the FGFR agonist is selected from the group consisting of FGF2, SUN11602, FGF1, FGF3, FGF4, FGF5, FGF6, FGF8, FGF10, FGF17, FGF19, FGF20, FGF21, FGF22, and FGF23. In some embodiments, the FGFR agonist is FGF2. In some embodiments, FGF2 is present in the culture medium at a concentration of 2-150 ng/ml.

In some embodiments, the c-kit agonist is SCF. In some embodiments, SCF is present in the culture medium at a concentration of 2-200 ng/ml.

In some embodiments, the IL-3R agonist is selected from the group consisting of IL-3, SC-65461, SC-55494 (Synthokine 1), and a peptide derived from IL-3. In some embodiments, the IL-3R agonist is IL-3. In some embodiments, IL-3 is present in the culture medium at a concentration of 0.02-100 ng/ml.

In some embodiments, the antioxidant is selected from the group consisting of lipoic acid, N-acetyl-L-Cysteine, ascorbic acid, sodium ascorbate, glutathione, ebselen, α-tocopherol, β-tocopherol, δ-tocopherol, γ-tocopherol, uric acid, and ubiquinol. In some embodiments, the antioxidant is lipoic acid. In some embodiments, lipoic acid is present in the culture medium at a concentration of 1-260 μM.

In some embodiments, the FLT3 agonist is selected from the group consisting of FLT3L, CDX-301, GS-3583, FLT3L-Fc, and a peptide derived from FLT3L. In some embodiments, the FLT3 agonist is FLT3L. In some embodiments, FLT3L is present in the culture medium at a concentration of 0.2-200 ng/ml.

In some embodiments, the TGF-β antagonist is selected from the group consisting of SB431542, A 83-01, GW788388, SB525334, SB505124, TP0427736, RepSox, SD-208, Galunisertib, IN-1130, LY2109761, LY550410, and TEW-7197. In some embodiments, the TGF-β antagonist is SB431542. In some embodiments, SB431542 is present in the culture medium at a concentration of 0.2-50 μM.

In some embodiments, the TRPV4 agonist is selected from the group consisting of GSK1016790A, 4α-Phorbol 12,13-didecanoate, N-Arachidonoyl Taurine, and RN-1747. In some embodiments, the TPRV4 agonist is GSK1016790A. In some embodiments, GSK1016790A is present in the culture medium at a concentration of 0.2-500 nM.

In some embodiments, the bioactive phospholipid is selected from the group consisting of sphingosine-1-phosphage (S1P), lysophosphatidic acid (LPA), ceramide-1-phosphate (C1P), and lysophosphatidylcholine (LPC). In some embodiments, the bioactive phospholipid is S1P. In some embodiments, S1P is present in the culture medium at a concentration of 0.005-1 μM.

In some embodiments, the IGF-1 receptor agonist is selected from the group consisting of IGF-1, IGF-1-Ado, X10, Mecasermin, IGF-2, Insulin, Rg5, IGF-1 24-41, IGF-1 30-41, des(1-3)IGF-1, IGF-1 LR3, and Demethylasterriquinone B1. In some embodiments, the IGF-1 receptor agonist is IGF-1. In some embodiments, IGF-1 is present in the culture medium at a concentration of 0.2-200 ng/ml.

In some embodiments, the method of generating a population of human CD34+, CD43+, and CD90+ HPCs comprises culturing a population of human CD34+ HSCs in a culture medium comprising VEGF, FGF2, SCF, IL-3, lipoic acid, FLT3L, SB431542, GSK1016790A, S1P, and IGF-1. In some embodiments, VEGF is present at a concentration of 20 ng/ml, FGF2 is present at a concentration of 10 ng/ml, SCF is present at a concentration of 15 ng/ml, IL-3 is present at a concentration of 10 ng/ml, lipoic acid is present at a concentration of 130 μM, FLT3L is present at a concentration of 20 ng/ml, SB431542 is present at a concentration of 4.5 μM, GSK1016790A is present at a concentration of 25 nM, S1P is present at a concentration of 40 nM, and IGF-1 is present at a concentration of 30 ng/ml.

In another aspect, the disclosure pertains to an isolated cell culture comprising at least 100 million human HPCs in a culture medium comprising a VEGFR agonist, an FGFR agonist, a c-kit agonist, a TPOR agonist, and a FLT3 agonist.

In some embodiments, the HPCs are CD34+, CD43+, and CD90+.

In some embodiments, after 42-54 hours of culture, the HPCs express normalized gene levels of HLF in a range of 35-140, MECOM in a range of 140-560, SPINK2 in a range of 25-100, CD45 in a range of 43-172, and MLLT3 in a range of 250-1000; after 66-78 hours of culture, the HPCs express normalized gene levels of HLF in a range of 25-110, MECOM in a range of 47-188, SPINK2 in a range of 50-200, CD45 in a range of 158-632, and MLLT3 in a range of 180-720; and after 90-102 hours of culture, the HPCs express normalized gene levels of HLF in a range of 6-24, MECOM in a range of 22-90, SPINK2 in a range of 50-100, CD45 in a range of 162-648, and MLLT3 in a range of 163-652.

In some embodiments, the HPCs possess, per million cells plated, in a colony-forming unit assay: after 42-54 hours of culture, 1000-7000 burst forming units for erythroid potential; 4000-7000 forming units for erythroid potential; 0-1000 colony-forming units for macrophage potential; 0-3000 colony-forming units for granulocyte potential; 500-2500 colony-forming units for macrophage-granulocyte potential; and 0-2000 colony-forming units for granulocyte-erythroid-macrophage-megakaryocyte potential; after 66-78 hours of culture, 500-2000 burst forming units for erythroid potential; 4000-6000 colony-forming units for erythroid potential; 0-1000 colony-forming units for macrophage potential; 0-3000 colony-forming units for granulocyte potential; 0-4000 colony-forming units for macrophage-granulocyte potential; and 0-2000 colony-forming units for granulocyte-erythroid-macrophage-megakaryocyte potential; and after 90-102 hours of culture, 500-1000 burst forming units for erythroid potential; 1000-4000 colony-forming units for erythroid potential; 0-100 colony-forming units for macrophage potential; 0-3000 colony-forming units for granulocyte potential; 0-4000 colony-forming units for macrophage-granulocyte potential; and 0-1000 colony-forming units for granulocyte-erythroid-macrophage-megakaryocyte potential.

In some embodiments, the HPCs are differentiated from induced pluripotent stem cells (IPSCs).

In some embodiments, the HPCs are derived from umbilical cord blood.

In some embodiments, the HPCs are derived from bone marrow.

In some embodiments, the HPCs are genetically engineered.

In another aspect, the disclosure pertains to an isolated cell culture comprising at least 100 million human HPCs in a culture medium comprising a VEGFR agonist, an FGFR agonist, a c-kit agonist, an IL-3R agonist, an antioxidant, a FLT3 agonist, a TGF-β antagonist, a TRPV4 agonist, a bioactive phospholipid, and an IGF-1 receptor agonist.

In some embodiments, the HPCs are CD34+, CD43+, and CD90+.

In some embodiments, after 42-54 hours of culture, the HPCs express normalized gene levels of HLF in a range of 15-66, MECOM in a range of 80-316, SPINK2 in a range of 15-60, CD45 in a range of 43-172, and MLLT3 in a range of 274-1096; after 66-78 hours of culture, the HPCs express normalized gene levels of HLF in a range of 55-220, MECOM in a range of 73-292, SPINK2 in a range of 50-200, CD45 in a range of 305-1200, and MLLT3 in a range of 223-930; and after 90-102 hours of culture, the HPCs express normalized gene levels of HLF in a range of 40-160, MECOM in a range of 65-262, SPINK2 in a range of 178-712, CD45 in a range of 625-2500, and MLLT3 in a range of 208-834.

In some embodiments, the HPCs possess, per million cells plated, in a colony-forming unit assay: after 42-54 hours of culture, 1500-7000 burst forming units for erythroid potential; 4000-7000 colony-forming units for erythroid potential; 0-1000 colony-forming units for macrophage potential; 0-3000 colony-forming units for granulocyte potential; 500-2500 colony-forming units for macrophage-granulocyte potential; and 0-2000 colony-forming units for granulocyte-erythroid-macrophage-megakaryocyte potential; after 66-78 hours of culture, 500-2000 burst forming units for erythroid potential; 4000-6000 colony-forming units for erythroid potential; 0-1000 colony-forming units for macrophage potential; 0-3000 colony-forming units for granulocyte potential; 0-4000 colony-forming units for macrophage-granulocyte potential; and 0-2000 colony-forming units for granulocyte-erythroid-macrophage-megakaryocyte potential; and after 90-102 hours of culture, 500-1000 burst forming units for erythroid potential; 1000-4000 colony-forming units for erythroid potential; 0-100 colony-forming units for macrophage potential; 0-3000 colony-forming units for granulocyte potential; 0-4000 colony-forming units for macrophage-granulocyte potential; and 0-1000 colony-forming units for granulocyte-erythroid-macrophage-megakaryocyte potential.

In some embodiments, the HPCs are differentiated from iPSCs.

In some embodiments, the HPCs are derived from umbilical cord blood.

In some embodiments, the HPCs are derived from bone marrow.

In some embodiments, the HPCs are genetically engineered.

Other features and advantages of the invention will be apparent from the following detailed description and claims.

### BRIEF DESCRIPTION OF THE DRAWINGS

FIG. 1 illustrates the differentiation process for induced pluripotent stem cell (iPSC)-derived induced hematopoietic progenitor cells (iHPCs). Stage 1 refers to the differentiation of human iPSCs (hiPSCs) into mesodermal cells that express MIXL1 and KDR. Stage 2 refers to the differentiation of the mesodermal cells into hemogenic endothelial cells that express CD31, CD34, KDR, and RUNX1. Stage 3 refers to the differentiation of the hemogenic endothelial cells into early iHPCs that express CD34, CD43, and CD90. Early iHPCs develop into middle iHPCs then late iHPCs, both of which also express CD34, CD43, and CD90.

FIG. 2 shows the percentage of the early, middle, and late iHPCs generated with Recipe 1 and Recipe 2 that are positive for CD34, CD43, CD90, CD38, or CD45RA.

FIG. 3A shows the changes in the normalized gene expressions of HLF, MECOM, SPINK2, MLLT3, RUNX1, and CD45 in the iHPCs generated with Recipe 1 and Recipe 2, as the iHPCs develop from early stage to middle and late stages.

FIG. 3B is a dimensionality reduction plot (PCA) showing the transcriptomic analysis of the cells generated with Recipe 1 (R1) and Recipe 2 (R2) at Stage 0 (S0), Stage 2 (S2) and Stage 3 (S3).

FIG. 4 shows the percentage of the early, middle, and late iHPCs that are viable at post-thaw.

FIG. 5 shows the number of burst forming units for erythroid potential (BFU-E), colony-forming units for erythroid potential (CFU-E), colony-forming units for macrophage potential (CFU-M), colony-forming units for granulocyte potential (CFU-G), colony-forming units for macrophage-granulocyte potential (CFU-GM), and colony-forming units for granulocyte-erythroid-macrophage-megakaryocyte potential (CFU-GEMM) from the early, middle, and late iHPCs generated with Recipe 1 and Recipe 2.

FIGS. 6A-6D are a set of flow cytometry plots showing the differentiation of the early, middle, and late iHPCs generated by Recipe 1 and Recipe 2 into monocyte, neutrophil, erythroid, and T-cell lineages, respectively.

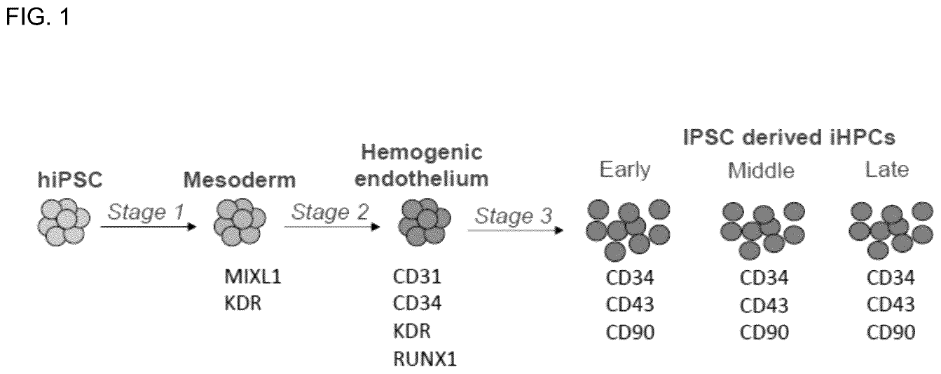

### DETAILED DESCRIPTION OF THE INVENTION

Described herein are methodologies and compositions that allow for generation of human hematopoietic progenitor cells (HPCs) from human hematopoietic stem cells (HSCs) under chemically-defined culture conditions using a small molecule based approach. The High-Dimensional Design of Experiments (HD-DoE) approach was used to simultaneously test multiple process inputs (e.g., small molecule agonists or antagonists) on output responses, such as gene expression. This approach allowed for the identification of chemically-defined culture media, comprising agonists and/or antagonists of particular signaling pathways, that are sufficient to generate HPCs in a very short amount of time. As described herein, the inventors have discovered that a population of CD34+, CD43+, and CD90+ HPCs can be generated from induced pluripotent stem cells (iPSCs) in as little as six days. In particular, the optimized culture media provided herein can be used to generate a population of CD34+, CD43+, and CD90+ HPCs from HSCs in as little as 12 hours. Flow cytometry analysis further confirmed the phenotype of the cells generated by the differentiation protocol. Various aspects of the invention are described in further detail in the following subsections.

### I. Cells

The starting cells used in the cultures of the disclosure can be human HSCs. As used herein, the terms “hematopoietic stem cell” and “HSC” refer to a stem cell that has the capacity to differentiate into a variety of different hematopoietic cell types. CD34 is a transmembrane phosphoglycoprotein that has been established in the art as a surface marker for HSCs. Human HSCs can be obtained from human umbilical cord blood and adult bone marrow.

HSCs are subjected to culture conditions, as described herein, that induce cellular differentiation. As used herein, the term “differentiation” refers to the development of a cell from a more primitive stage towards a more mature (i.e. less primitive) cell, typically exhibiting phenotypic features of commitment to a particular cellular lineage.

In some embodiments, cells can be identified and characterized based on expression of one or more biomarkers. A “positive” biomarker is one that is expressed on a cell of interest, whereas a “negative” biomarker is one that is not expressed on a cell of interest. Non-limiting examples of biomarkers whose expression can be evaluated in the characterization of a population of HPCs generated by the methods of the disclosure are HLF, MECOM, SPINK2, MLLT3, RUNX1, and CD45.

As used herein, expression by a cell of only “low” levels of a biomarker of interest is intended to refer to a level that is at most 20%, and more preferably, less than 20%, less than 15%, less than 10%, or less than 5% above background levels (wherein background levels correspond to, for example, the level of expression of a negative control marker that is considered to not be expressed by the cell).

In some embodiments, the cells generated by the methods of the disclosure are HPCs. As used herein, the terms “hematopoietic progenitor cell” and “HPC” refer to a cell that is more differentiated than an HSC and that can be further differentiated into a particular type of hematopoietic cell. In some embodiments, HPCs are generated from induced pluripotent stem cells (iPSCs). In some embodiments, HPCs are derived from umbilical cord blood or bone marrow. In some embodiments, HPCs are genetically engineered. HPCs may express at least one biomarker selected from HLF, MECOM, SPINK2, MLLT3, RUNX1, and CD45. In some embodiments, HPCs express CD34, CD43, and CD90.

As used herein, the term “engineered”cells refers to cells that are not naturally-occurring. Engineered cells may be result from genetic engineering, which modifies cells to contain and/or express a foreign gene or nucleic acid sequence, which, in turn, modifies the genotype and/or phenotype of the cells or their progeny. Engineered cells may also be obtained from preconditioning, biomaterial encapsulation, surface modification, or cell assembly approaches. For stem cells, engineered cells may be obtained from a directed differentiation process that involves external factors.

### Il. Culture Media Components

The methods of the disclosure for generating human CD34+, CD43+, and CD90+ HPCs comprise culturing human CD34+HSCs in a culture medium comprising specific agonist and/or antagonists of cellular receptors and/or signaling pathways. A culture medium comprising a VEGFR agonist, an FGFR agonist, a c-kit agonist, a TPOR agonist, and a FLT3 agonist was sufficient to generate a population of human CD34+, CD43+, and CD90+ HPCs. A culture medium comprising a VEGFR agonist, an FGFR agonist, a c-kit agonist, an IL-3R agonist, an antioxidant, a FLT3 agonist, a TGF-β antagonist, a TRPV4 agonist, a bioactive phospholipid, and an IGF-1 receptor agonist was also developed for generating a population of human CD34+, CD43+, and CD90+ HPCs.

As used herein, the term “agonist” refers to an agent that stimulates (upregulates) the cellular receptor or signaling pathway. Stimulation of the cellular signaling pathway can be initiated extracellularly, for example by use of an agonist that activates a cell surface receptor involved in the signaling pathway (e.g., the agonist can be a receptor ligand). Additionally or alternatively, stimulation of cellular signaling can be initiated intracellularly, for example by use of a small molecule agonist that interacts intracellularly with a component(s) of the signaling pathway.

As used herein, the term “antagonist” refers to an agent that inhibits (downregulates) the cellular signaling pathway. Inhibition of the cellular signaling pathway can be initiated extracellularly, for example by use of an antagonist that blocks a cell surface receptor involved in the signaling pathway. Additionally or alternatively, inhibition of cellular signaling can be initiated intracellularly, for example by use of a small molecule antagonist that interacts intracellularly with a component(s) of the signaling pathway.

Agonists and antagonists used in the methods of the disclosure are known in the art and commercially available. They are used in the culture media at a concentration effective to achieve the desired outcome, e.g., generation of HPCs expressing markers of interest. Non-limiting examples of suitable agonistic and antagonistic agents, and effective concentration ranges, are described further below.

As used herein, the terms “vascular endothelial growth factor receptor agonist” and “VEGFR agonist” refer to an agent that stimulates or increases signaling through or activity of a VEGFR (e.g., VEGFR1, VEGFR2, and VEGFR3). In some embodiments, the VEGFR agonist binds to a VEGFR and stimulates or increases the activity of or through the VEGFR. In some examples, a VEGFR agonist is capable of (i) increasing phosphorylation of VEGFRs, (ii) increasing expression of VEGF protein levels, or (iii) increasing expression of VEGF mRNA levels. In some embodiments, the VEGFR agonist is VEGF (e.g., VEGF-A, VEGF-B, VEGF-C, VEGF-D, and VEGF-E). In other embodiments, the VEGFR agonist is Gremlin, PG-007, Taurocholic acid, KLTWQELYQLKYKGI, or a peptide derived from VEGF. In one embodiment, the VEGFR agonist is present in the culture medium at a concentration of 1-400 ng/ml, 1-30 ng/ml, 25-55 ng/ml, 50-80 ng/ml, 75-105 ng/ml, 100-130 ng/ml, 125-155 ng/ml, 150-180 ng/ml, 175-205 ng/ml, 200-230 ng/ml, 225-255 ng/ml, 250-280 ng/ml, 275-305 ng/ml, 300-330 ng/ml, 325-355 ng/ml, 350-380 ng/ml, or 375-400 ng/ml. In one embodiment, the VEGFR agonist is VEGF. In one embodiment, the VEGFR agonist is VEGF, which is present in the culture medium at a concentration of 1-400 ng/ml, 1-30 ng/ml, 25-55 ng/ml, 50-80 ng/ml, 75-105 ng/ml, 100-130 ng/ml, 125-155 ng/ml, 150-180 ng/ml, 175-205 ng/ml, 200-230 ng/ml, 225-255 ng/ml, 250-280 ng/ml, 275-305 ng/ml, 300-330 ng/ml, 325-355 ng/ml, 350-380 ng/ml, or 375-400 ng/ml. In one embodiment, the VEGFR agonist is VEGF, which is present in the culture medium at a concentration of 20 ng/ml.

As used herein, the terms “fibroblast growth factor receptor agonist” and “FGFR agonist” refer to an agent that stimulates or increases signaling through or activity of an FGFR. In some examples, an FGFR agonist is capable of (i) increasing the phosphorylation of FRS2a, (ii) increasing phosphorylation of ERK, or (iii) increasing phosphorylation of AKT. In some embodiments, the FGFR agonist binds an FGFR and stimulates or increases signaling through or activity of the FGFR. In some embodiments, the FGFR agonist is a naturally-occurring ligand. In some embodiments, the naturally-occurring ligand of FGFR is FGF2, FGF1, FGF3, FGF4, FGF5, FGF6, FGF8, FGF10, FGF17, FGF19, FGF20, FGF21, FGF22, or FGF23. In some embodiments, the FGFR agonist is a ligand mimetic. In some embodiments, the FGFR ligand mimetic is SUN11602. In some embodiments, the FGFR agonist is FGF2. In other embodiments, the FGFR agonist is SUN11602, FGF1, FGF3, FGF4, FGF5, FGF6, FGF8, FGF10, FGF17, FGF19, FGF20, FGF21, FGF22, or FGF23. In one embodiment, the FGFR agonist is present in the culture medium at a concentration of 2-150 ng/ml, 2-20 ng/ml, 15-35 ng/ml, 30-50 ng/ml, 45-65 ng/ml, 60-80 ng/ml, 75-95 ng/ml, 90-110 ng/ml, 105-125 ng/ml, 120-140 ng/ml, or 135-150 ng/ml. In one embodiment, the FGFR agonist is FGF2. In one embodiment, the FGFR agonist is FGF2, which is present in the culture medium at a concentration of 2-150 ng/ml, 2-20 ng/ml, 15-35 ng/ml, 30-50 ng/ml, 45-65 ng/ml, 60-80 ng/ml, 75-95 ng/ml, 90-110 ng/ml, 105-125 ng/ml, 120-140 ng/ml, or 135-150 ng/ml. In one embodiment, the FGFR agonist is FGF2, which is present in the culture medium at a concentration of 10 ng/ml.

As used herein, the term “C-kit agonist” refers to an agent that stimulates or increases signaling through or activity of a c-kit receptor (CD117). In some examples, a c-kit agonist is capable of (i) increasing the expression of Bcl-2 protein, (ii) increasing SCF-induced phosphorylation of AKT, or (iii) increasing SCF-induced phosphorylation of ERK. In some embodiments, the c-kit agonist is a c-kit ligand. In one embodiment, the c-kit agonist is present in the culture medium at a concentration of 2-200 ng/ml, 2-20 ng/ml, 15-35 ng/ml, 30-50 ng/ml, 45-65 ng/ml, 60-80 ng/ml, 75-95 ng/ml, 90-110 ng/ml, 105-125 ng/ml, 120-140 ng/ml, 135-150 ng/ml, 145-165 ng/ml, 160-180 ng/ml, or 175-200 ng/ml. In some embodiments, the c-kit ligand is stem cell factor (SCF). In one embodiment, SCF is present in the culture medium at a concentration of 2-200 ng/ml, 2-20 ng/ml, 15-35 ng/ml, 30-50 ng/ml, 45-65 ng/ml, 60-80 ng/ml, 75-95 ng/ml, 90-110 ng/ml, 105-125 ng/ml, 120-140 ng/ml, 135-150 ng/ml, 145-165 ng/ml, 160-180 ng/ml, or 175-200 ng/ml. In one embodiment, SCF is present in the culture medium at a concentration of 50 ng/ml. In one embodiment, SCF is present in the culture medium at a concentration of 15 ng/ml.

As used herein, the terms “thrombopoietin receptor agonist” and “TPOR agonist” refer to an agent that stimulates or increases signaling through or activity of an TPOR. In some examples, a TPOR agonist is capable of (i) increasing phosphorylation of JAK2 or (ii) increasing phosphorylation of STAT5. In some embodiments, the TPOR agonist is thrombopoietin (TPO). In other embodiments, the TPOR agonist is eltrombopag, TA-316, TPO agonist 1, avatrombopag, or lusutrombopag. In one embodiment, the TPOR agonist is present in the culture medium at a concentration of 2-20 ng/ml, 15-35 ng/ml, 30-50 ng/ml, 45-65 ng/ml, 60-80 ng/ml, 75-95 ng/ml, 90-110 ng/ml, 105-125 ng/ml, 120-140 ng/ml, 135-150 ng/ml, 145-165 ng/ml, 160-180 ng/ml, or 175-200 ng/ml. In one embodiment, the TPOR agonist is TPO. In one embodiment, the TPOR agonist is TPO, which is present in the culture medium at a concentration of 1-200 ng/ml, 2-20 ng/ml, 15-35 ng/ml, 30-50 ng/ml, 45-65 ng/ml, 60-80 ng/ml, 75-95 ng/ml, 90-110 ng/ml, 105-125 ng/ml, 120-140 ng/ml, 135-150 ng/ml, 145-165 ng/ml, 160-180 ng/ml, or 175-200 ng/ml. In one embodiment, the TPOR agonist is TPO, which is present in the culture medium at a concentration of 20 ng/ml.

As used herein, the terms “fms-like tyrosine kinase 3 agonist” and “FLT3 agonist” refer to an agent that stimulates or increases signaling through or activity of a FLT3 (CD135). In some examples, a FLT3 agonist is capable of (i) increasing phosphorylation of FLT or (ii) increasing expression of FLT3 protein levels. In some embodiments, the FLT3 agonist binds to a FLT3 and stimulates or increases the activity of or through the FLT3. In some embodiments, the FLT3 agonist is Flt3 ligand (FLT3L). In other embodiments, the FLT3 agonist is CDX-301, GS-3583, FLT3L-Fc, or a peptide derived from FLT3L. In one embodiment, the FLT3 agonist is present in the culture medium at a concentration of 0.2-200 ng/ml, 1-200 ng/ml, 0.2-1 ng/ml, 0.5-1.5 ng/ml, 1-20 ng/ml, 15-35 ng/ml, 30-50 ng/ml, 45-65 ng/ml, 60-80 ng/ml, 75-95 ng/ml, 90-110 ng/ml, 105-125 ng/ml, 120-140 ng/ml, 135-150 ng/ml, 145-165 ng/ml, 160-180 ng/ml, or 175-200 ng/ml. In one embodiment, the FLT3 agonist is FLT3L. In one embodiment, the FLT3 agonist is FLT3L, which is present in the culture medium at a concentration of 0.2-200 ng/ml, 1-200 ng/ml, 0.2-1 ng/ml, 0.5-1.5 ng/ml, 1-20 ng/ml, 15-35 ng/ml, 30-50 ng/ml, 45-65 ng/ml, 60-80 ng/ml, 75-95 ng/ml, 90-110 ng/ml, 105-125 ng/ml, 120-140 ng/ml, 135-150 ng/ml, 145-165 ng/ml, 160-180 ng/ml, or 175-200 ng/ml. In one embodiment, the FLT3 agonist is FLT3L, which is present in the culture medium at a concentration of 20 ng/ml.

As used herein, the terms “interleukin 3 receptor agonist” and “IL-3R agonist” refer to an agent that stimulates or increases signaling through or activity of an IL-3R (CD123). In some examples, an IL-3R agonist is capable of (i) increasing phosphorylation of JAKs, (ii) increasing phosphorylation of STATS, or (iii) increasing expression of c-myc. In some embodiments, the IL-3R agonist binds to an IL-3R and stimulates or increases the activity of or through the IL-3R. In some embodiments, the IL-3R agonist is IL-3. In other embodiments, the IL-3R agonist is SC-65461, SC-55494 (Synthokine 1), or a peptide derived from IL-3. In one embodiment, the IL-3R agonist is present in the culture medium at a concentration of 0.02-100 ng/ml, 0.02-0.07 ng/ml, 0.05-0.1 ng/ml, 0.75-1.25 ng/ml, 1-10 ng/ml, 5-15 ng/ml, 10-20 ng/ml, 15-25 ng/ml, 20-30 ng/ml, 25-35 ng/ml, 30-40 ng/ml, 35-45 ng/ml, 40-50 ng/ml, 45-55 ng/ml, 50-60 ng/ml, 55-65 ng/ml, 60-70 ng/ml, 65-75 ng/ml, 70-80 ng/ml, 75-85 ng/ml, 80-90 ng/ml, or 85-100 ng/ml. In one embodiment, the IL-3R agonist is IL-3. In one embodiment, the IL-3R agonist is IL-3, which is present in the culture medium at a concentration of 0.02-100 ng/ml, 0.02-0.07 ng/ml, 0.05-0.1 ng/ml, 0.75-1.25 ng/ml, 1-10 ng/ml, 5-15 ng/ml, 10-20 ng/ml, 15-25 ng/ml, 20-30 ng/ml, 25-35 ng/ml, 30-40 ng/ml, 35-45 ng/ml, 40-50 ng/ml, 45-55 ng/ml, 50-60 ng/ml, 55-65 ng/ml, 60-70 ng/ml, 65-75 ng/ml, 70-80 ng/ml, 75-85 ng/ml, 80-90 ng/ml, or 85-100 ng/ml. In one embodiment, the IL-3R agonist is IL-3, which is present in the culture medium at a concentration of 10 ng/ml.

As used herein, the term “antioxidant” refers to an antioxidant that inhibits or decreases oxidation which produces free radicals. In some examples, an antioxidant is capable of (i) decreasing expression of reactive oxygen species (ROS), (ii) inhibiting ROS-induced expression of caspases, or (iii) decreasing expression of reactive nitrogen compounds. In some embodiments, the antioxidant directly reacts with free radicals. In some embodiments, the antioxidant inhibits the activity or expression of free radical generating enzymes. In some embodiments, the antioxidant stimulates the activity or expression of intracellular antioxidant enzymes. In some embodiments, the antioxidant is lipoic acid. In other embodiments, the antioxidant is N-acetyl-L-Cysteine, ascorbic acid, sodium ascorbate, glutathione, ebselen, α-tocopherol, β-tocopherol, δ-tocopherol, γ-tocopherol, uric acid, or ubiquinol. In one embodiment, the antioxidant is present in the culture medium at a concentration of 1-260 μM, 1-20 μM, 15-35 μM, 30-50 μM, 45-65 μM, 60-80 μM, 75-95 μM, 90-110 μM, 105-125 μM, 120-140 μM, 135-55 μM, 150-170 μM, 165-185 μM, 180-200 μM, 195-215 μM, 210-230 μM, 225-245 μM, or 240-260 μM. In one embodiment, the antioxidant is lipoic acid. In one embodiment, the antioxidant is lipoic acid, which is present in the culture medium at a concentration of 1-260 μM, 1-20 μM, 15-35 μM, 30-50 μM, 45-65 μM, 60-80 μM, 75-95 μM, 90-110 μM, 105-125 μM, 120-140 μM, 135-155 μM, 150-170 μM, 165-185 μM, 180-200 μM, 195-215 μM, 210-230 μM, 225-245 μM, or 240-260 μM. In one embodiment, the antioxidant is lipoic acid, which is present in the culture medium at a concentration of 130 μM.

As used herein, the terms “transforming growth factor beta antagonist” and “TGF-β antagonists” refer to an agent that inhibit or decrease signaling through or activity of a TGF-β receptor family member. In some examples, a TGF-β antagonist is capable of (i) reducing phosphorylation of the type I receptor or (ii) reducing phosphorylation of SMAD 2/3. In some embodiments, the TGF-β antagonist binds to a type II or type I receptor and inhibits or decreases signaling through or activity of the type II or type I receptor. In some embodiments, the TGF-β antagonist is SB431542. In other embodiments, the TGF-β antagonist is A 83-01, GW788388, SB525334, SB505124, TP0427736, RepSox, SD-208, Galunisertib, IN-1130, LY2109761, LY550410, or TEW-7197. In one embodiment, the TGF-β antagonist is present in the culture medium at a concentration of 0.2-50 μM, 0.2-1.2 μM, 1-10 μM, 5-15 μM, 10-20 μM, 15-25 μM, 20-30 μM, 25-35 μM, 30-40 μM, 35-45 μM, or 40-50 μM. In one embodiment, the TGF-β antagonist is SB431542. In one embodiment, the TGF-β antagonist is SB431542, which is present in the culture medium at a concentration of 0.2-50 μM, 0.2-1.2 μM, 1-10 μM, 5-15 μM, 10-20 μM , 15-25 μM, 20-30 μM, 25-35 μM, 30-40 μM, 35-45 μM, or 40-50 μM. In one embodiment, the TGF-β antagonist is SB431542, which is present in the culture medium at a concentration of 4.5 μM.

As used herein, the terms “transient receptor potential vanilloid 4 agonist” and “TRPV4 agonist” refer to an agent that stimulates or increases signaling through or activity of the TRPV4 signaling pathway. In some examples, a TRPV4 agonist is capable of increasing the intracellular concentration of Ca2+. In some embodiments, the TRPV4 agonist binds to a TRPV4 channel and stimulates or increases the activity of or through the TRPV4. In one embodiment, the TRPV4 agonist is present in the culture medium at the concentration of 0.2-500 nM, 0.2-30 nM, 25-55 nM, 50-80 nM, 75-105 nM, 100-130 nM, 125-155 nM, 150-180 nM, 175-205 nM, 200-230 nM, 225-255 nM, 250-280 nM, 275-305 nM, 300-330 nM, 325-355 nM, 350-380 nM, 375-405 nM, 400-430 nM, 425-455 nM, 450-480 nM, or 475-500 nM. In some embodiments, the TRPV4 agonist is GSK1016790A. In other embodiments, the TRPV4 agonist is 4α-Phorbol 12,13-didecanoate, N-Arachidonoyl Taurine or RN-1747. In one embodiment, the TRPV4 agonist is GSK1016790A. In one embodiment, the TRPV4 agonist is GSK1016790A, which is present in the culture medium at a concentration of 0.2-500 nM, 0.2-30 nM, 25-55 nM, 50-80 nM, 75-105 nM, 100-130 nM, 125-155 nM, 150-180 nM, 175-205 nM, 200-230 nM, 225-255 nM, 250-280 nM, 275-305 nM, 300-330 nM, 325-355 nM, 350-380 nM, 375-405 nM, 400-430 nM, 425-455 nM, 450-480 nM, or 475-500 nM. In one embodiment, the TRPV4 agonist is GSK1016790A, which is present in the culture medium at a concentration of 25 nM.

In some embodiments, the bioactive phospholipid is sphingosine-1-phosphate (S1P). In other embodiments, the bioactive phospholipid is lysophosphatidic acid (LPA), ceramide-1-phosphate (C1P), or lysophosphatidylcholine (LPC). In one embodiment, the bioactive phospholipid is present in the culture medium at a concentration of 0.005-1 μM, 0.005-0.01 μM, 0.007-0.015 μM, 0.01-0.05 μM, 0.02-0.07 μM, 0.05-0.1 μM, 0.07-0.15 μM, 0.1-0.5 μM, 0.2-0.7 μM, or 0.5-1 μM. In one embodiment, the bioactive phospholipid is S1P. In one embodiment, the bioactive phospholipid is S1P, which is present in the culture medium at a concentration of 0.005-1 μM, 0.005-0.01 μM, 0.007-0.015 μM, 0.01-0.05 μM, 0.02-0.07 μM, 0.05-0.1 μM, 0.07-0.15 μM, 0.1-0.5 μM, 0.2-0.7 μM, or 0.5-1 μM. In one embodiment, the bioactive phospholipid is S1P, which is present in the culture medium at a concentration of 40 μM. In one embodiment, the bioactive phospholipid is S1P, which is present in the culture medium at a concentration of 40 nM.

As used herein, the terms “insulin-like growth factor 1 receptor agonist” and “IGF-1 receptor agonist” refer to an agent that stimulates or increases signaling through or activity of an IGF-1 receptor. In some examples, an IGF-1 receptor agonist is capable of (i) increasing IGF-induced phosphorylation of AKT or (ii) increasing IGF-induced phosphorylation of ERK. In an embodiment, the IGF-1 receptor agonist is IGF-1. In some embodiments, the IGF-1 receptor agonist binds to an IGF-1 receptor and stimulates or increases the activity of or through the IGF-1 receptor. In some embodiments, the IGF-1 receptor agonist is IGF-1. In other embodiments, the IGF-1 receptor agonist is IGF-1-Ado, X10, Mecasermin, IGF-2, Insulin, Rg5, IGF-1 24-41, IGF-1 30-41, des(1-3)IGF-1, IGF-1 LR3, or Demethylasterriquinone B1. In one embodiment, the IGF-1 receptor agonist is present in the culture medium at a concentration of 0.2-200 ng/ml, 0.2-1 ng/ml, 0.5-1.5 ng/ml, 1-20 ng/ml, 15-35 ng/ml, 30-50 ng/ml, 45-65 ng/ml, 60-80 ng/ml, 75-95 ng/ml, 90-110 ng/ml, 105-125 ng/ml, 120-140 ng/ml, 135-155 ng/ml, 150-170 ng/ml, 165-185 ng/ml, or 180-200 ng/ml. In one embodiment, the IGF-1 receptor agonist is IGF-1. In one embodiment, the IGF-1receptor agonist is IGF-1, which is present in the culture medium at a concentration of 0.2-200 ng/ml, 0.2-1 ng/ml, 0.5-1.5 ng/ml, 1-20 ng/ml, 15-35 ng/ml, 30-50 ng/ml, 45-65 ng/ml, 60-80 ng/ml, 75-95 ng/ml, 90-110 ng/ml, 105-125 ng/ml, 120-140 ng/ml, 135-155 ng/ml, 150-170 ng/ml, 165-185 ng/ml, or 180-200 ng/ml. In one embodiment, the IGF-1 receptor agonist is IGF-1, which is present in the culture medium at a concentration of 30 ng/ml.

In some embodiments, the methods of generating a population of human CD34+, CD43+, and CD90+ HPCs comprise culturing a population of human CD34+ HSCs in a culture medium comprising VEGF, FGF2, SCF, TPO, and FLT3L for at least 12 hours. In one embodiment, these agents are present in the culture medium at a concentration as set forth above. In one embodiment, VEGF is present at a concentration of 20 ng/ml, FGF2 is present at a concentration of 10 ng/ml, SCF is present at a concentration of 50 ng/ml, TPO is present at a concentration of 20 ng/ml, and FLT3L is present at a concentration of 20 ng/ml.

In some embodiments, the methods of generating a population of human CD34+, CD43+, and CD90+ HPCs comprise culturing a population of human CD34+ HSCs in a culture medium comprising VEGF, FGF2, SCF, IL-3, lipoic acid, FLT3L, SB431542, GSK1016790A, S1P, and IGF-1 for at least 12 hours. In one embodiment, these agents are present in the culture medium at a concentration as set forth above. In one embodiment, VEGF is present at a concentration of 20 ng/ml, FGF2 is present at a concentration of 10 ng/ml, SCF is present at a concentration of 15 ng/ml, IL-3 is present at a concentration of 10 ng/ml, lipoic acid is present at a concentration of 130 μM, FLT3L is present at a concentration of 20 ng/ml, SB431542 is present at a concentration of 4.5 μM, GSK1016790A is present at a concentration of 25 nM, S1P is present at a concentration of 40 nM, and IGF-1 is present at a concentration of 30 ng/ml.

### III. Culture Conditions

In combination with the chemically-defined and optimized culture media described in subsection II above, the methods of generating human CD34+, CD43+, and CD90+ HPCs of the disclosure utilize standard culture conditions established in the art for cell culture. For example, cells can be cultured at 37° C. and under 5% O₂ and 5% CO₂ conditions. A basal media can be used as the starting media to which supplemental agents can be added. For example, in one embodiment, the commercially available StemSpan™ SFEM II media is used as basal media. Cells can be cultured in standard culture vessels or plates, such as culture dishes, culture flasks, or 96-well plates.

The culture media typically are changed regularly to fresh media. For example, in one embodiment, the culture medium is changed every 72 hours.

The starting human CD34+ HSCs can be obtained by methodologies established in the art.

Sources of human CD34+ HSCs include umbilical cord blood and bone marrow. Human CD34+ HSCs can be obtained, for example, by standard magnetic enrichment.

To generate human CD34+, CD43+, and CD90+ HPCs, the starting human HSCs are cultured in the optimized culture media for sufficient time. It has been discovered that culture of human CD34+ HSCs in the optimized culture media for as little as six days was sufficient for the generation of human CD34+, CD43+, and CD90+ HPCs.

In various embodiments, a population of human CD34+ HSCs is cultured in the optimized culture media for sufficient time to increase the expression of at least one, and preferably a plurality of, HPC-associated genetic markers. Non-limiting examples of suitable HPC-associated genetic markers are HLF, MECOM, SPINK2, MLLT3, RUNX1, and CD45. In some embodiments, cells are cultured for sufficient time to increase the expression levels of at least two, at least three, at least four, at least five, or at least six HPC-associated genetic markers. In one embodiment, cells are cultured for sufficient time to increase the expression level of at least one HPC-associated genetic marker (e.g., HLF) by at least 10%, 20%, 30%, 40%, 50%, 60%, 70%, 80%, 90%, or 100% as compared to the starting cell population. The level of expression of genetic markers in the cultured HPCs can be measured by techniques available in the art (e.g., RNAseq analysis).

In one embodiment, cells are cultured for at least 3 days, at least 4 days, at least 5 days, at least 6 days, at least 7 days, at least 10 days, at least 2 weeks, at least 3 weeks, at least 4 weeks, or longer.

### IV. Uses

The methods and compositions of the disclosure for generating human HPCs allow for efficient and robust availability of these cell populations for a variety of uses. For example, the methods and compositions can be used in the study of HPC development and differentiation, including biology to assist in the understanding of hematopoietic-related diseases and disorders. For example, the HPCs generated using the methods of the disclosure can be further purified according to methods established in the art using agents that bind to surface markers expressed on the cells.

Additionally, HPCs generated according to the methods of the disclosure are contemplated for use in the treatment of various hematopoietic diseases and disorders, through delivery of the cells to a subject having the disease or disorder (e.g., HPC transplantation). Hematopoietic diseases and disorders include, but are not limited to, cancers such as leukemias and lymphomas, blood disorders and autoimmune disorders.

### V. Compositions

In other aspects, the disclosure provides compositions related to the methods of generating human HPCs, including isolated cell cultures.

In one aspect, the disclosure provides an isolated cell culture of at least 100 million human CD34+, CD43+, and CD90+ HPCs in a culture medium comprising a VEGFR agonist, an FGFR agonist, a c-kit agonist, a TPOR agonist, and a FLT3 agonist. Suitable agents, and concentrations therefor, include those described in subsection II.

After 42-54 hours of culture, these HPCs express normalized gene levels of HLF in a range of 35-140, MECOM in a range of 140-560, SPINK2 in a range of 25-100, CD45 in a range of 43-172, and MLLT3 in a range of 250-1000. After 66-78 hours of culture, these HPCs express normalized gene levels of HLF in a range of 25-110, MECOM in a range of 47-188, SPINK2 in a range of 50-200, CD45 in a range of 158-632, and MLLT3 in a range of 180-720. Lastly, after 90-102 hours of culture, these HPCs express normalized gene levels of HLF in a range of 6-24, MECOM in a range of 22-90, SPINK2 in a range of 50-100, CD45 in a range of 162-648 and MLLT3 in a range of 163-652.

Additionally, these HPCs possess, per million cells plated, in a colony-forming unit assay: after 42-54 hours of culture, 1000-7000 burst forming units for erythroid potential; 4000-7000 colony-forming units for erythroid potential; 0-1000 colony-forming units for macrophage potential; 0-3000 colony-forming units for granulocyte potential; 500-2500 colony-forming units for macrophage-granulocyte potential; and 0-2000 colony-forming units for granulocyte-erythroid-macrophage-megakaryocyte potential; after 66-78 hours of culture, 500-2000 burst forming units for erythroid potential; 4000-6000 colony-forming units for erythroid potential; 0-1000 colony-forming units for macrophage potential; 0-3000 colony-forming units for granulocyte potential; 0-4000 colony-forming units for macrophage-granulocyte potential; and 0-2000 colony-forming units for granulocyte-erythroid-macrophage-megakaryocyte potential; and after 90-102 hours of culture, 500-1000 burst forming units for erythroid potential; 1000-4000 colony-forming units for erythroid potential; 0-100 colony-forming units for macrophage potential; 0-3000 colony-forming units for granulocyte potential; 0-4000 colony-forming units for macrophage-granulocyte potential; and 0-1000 colony-forming units for granulocyte-erythroid-macrophage-megakaryocyte potential.

In another aspect, the disclosure provides an isolated cell culture of a population of human CD34+, CD43+, and CD90+ HPCs in a culture medium comprising a VEGFR agonist, an FGFR agonist, a c-kit agonist, an IL-3R agonist, an antioxidant, a FLT3 agonist, a TGF-β antagonist, a TRPV4 agonist, a bioactive phospholipid, and an IGF-1 receptor agonist. Suitable agents, and concentrations therefor, include those described in subsection II.

After 42-54 hours of culture, these HPCs express normalized gene levels of HLF in a range of 15-66, MECOM in a range of 80-316, SPINK2 in a range of 15-60, CD45 in a range of 43-172, and MLLT3 in a range of 274-1096. After 66-78 hours of culture, these HPCs express normalized gene levels of HLF in a range of 55-220, MECOM in a range of 73-292, SPINK2 in a range of 50-200, CD45 in a range of 305-1200, and MLLT3 in a range of 223-930. After 90-102 hours of culture, these HPCs express normalized gene levels of HLF in a range of 40-160, MECOM in a range of 65-262, SPINK2 in a range of 178-712, CD45 in a range of 625-2500, and MLLT3 in a range of 208-834.

Additionally, these HPCs possess, per million cells plated, in a colony-forming unit assay: after 42-54 hours of culture, 1500-7000 burst forming units for erythroid potential; 4000-7000 colony-forming units for erythroid potential; 0-1000 colony-forming units for macrophage potential; 0-3000 colony-forming units for granulocyte potential; 500-2500 colony-forming units for macrophage-granulocyte potential; and 0-2000 colony-forming units for granulocyte-erythroid-macrophage-megakaryocyte potential; after 66-78 hours of culture, 500-2000 burst forming units for erythroid potential; 4000-6000 colony-forming units for erythroid potential; 0-1000 colony-forming units for macrophage potential; 0-3000 colony-forming units for granulocyte potential; 0-4000 colony-forming units for macrophage-granulocyte potential; and 0-2000 colony-forming units for granulocyte-erythroid-macrophage-megakaryocyte potential; and after 90-102 hours of culture, 500-1000 burst forming units for erythroid potential; 1000-4000 colony-forming units for erythroid potential; 0-100 colony-forming units for macrophage potential; 0-3000 colony-forming units for granulocyte potential; 0-4000 colony-forming units for macrophage-granulocyte potential; and 0-1000 colony-forming units for granulocyte-erythroid-macrophage-megakaryocyte potential.

### EXAMPLES

The following examples are put forth so as to provide those of ordinary skill in the art with a description of how the methods described herein may be used and evaluated and are intended to be purely exemplary of the invention and are not intended to limit the scope of what the inventors regard as their invention.

### Example 1

### Generation and Characterization of Hematopoietic Progenitor Cells

In this example, two culture medium recipes for the generation of CD34+, CD43+, and CD90+HPCs were developed. The recipes can be used to culture CD34+ cells, for example isolated from cord blood, and expand a population of HSCs.

The recipes were developed by utilizing High-Dimensional Design of Experiments (HD-DoE), as previously described in Bukys et al. (2020) Iscience 23:101346. The method employs computerized design geometries to simultaneously test multiple process inputs and offers mathematical modeling of a deep effector/response space. The method allows for finding combinatorial signaling inputs that control a complex process, such as during cell differentiation. It allows testing of multiple plausible critical process parameters, as such parameters impact output responses, such as gene expression. Because gene expression provides hallmark features of the phenotype of, for example, a human cell, the method can be applied to identify, and understand, which signaling pathways control cell fate. In the current example, the HD-DOE method was applied with the intent to find conditions for generating HPCs.

Specifically, to develop the HPC recipe, the impact of agonists and antagonists of multiple signaling pathways (herein called effectors) were tested on expression of two sets of 53 pre-selected genes after a three-day treatment. Specific genes in the measured subset are highly enriched in HPCs and include HLF, MECOM, SPINK2, MLLT3, RUNX1, and CD45. The effectors chosen include small molecules or proteins, some of which are used during in vitro expansion of HSCs.

### Characterization of HPCs

On day-2, induced pluripotent stem cells (iPSCs) were single cell seeded at 0.05729M cells/cm2 into vitronectin-coated (0.5 μg/cm2) flasks or plates in GIBCO E8 medium (ThermoFisher Cat #A2858501) containing a ROCK inhibitor.

On day −1, E8 medium was changed to remove the ROCK inhibitor.

On day 0, media was changed into Stage 1 medium for 48 hours for mesoderm commitment. The Stage 1 medium is CDM2 basal medium (50/50 IMDM/F12, 0.7 μg/ml insulin, 15 μg/ml transferrin, 1% chemically-defined lipid concentrate, 450 μM monothioglycerol, 1 mg/ml PVA) supplemented with 1× Penicillin-Streptomycin and 6 μM CHIR99021.

On day 2, cells were dissociated with accutase for 3 minutes and replated on vitronectin-coated (0.5 μg/cm2) flasks or plates in Stage 2 medium containing a ROCK inhibitor at a density of 0.08125M cells/cm2 for 24 hours for hemogenic endothelium commitment.

On day 3, Stage 2 medium was changed to remove the ROCK inhibitor and cultured for another 48 hours. The Stage 2 medium is as follows: GIBCO RPMI 1640 Medium, HEPES, 50 ng/ml VEGF, 10 ng/ml FGF2, 500 nM purmorphamine, 1 μM forskolin, 1 μg/ml thymosin B4, 20 ng/ml BMP4, 100 nM XAV939, 500 nM retinoic acid, 100 μg/ml ascorbic acid, 2% B-27 supplement, and 1× Penicillin-Streptomycin.

On day 5, Stage 2 cells underwent quality control by flow cytometry ensuring expression of hemogenic endothelial markers CD34, CD43, CD73, CD31, CD90, CXCR4, CD235, and RUNX1. Stage 2 cells were dissociated with accutase for three minutes and seeded into a PBS vertical wheel bioreactor at 0.257M cells/ml in either Stage 3A HPC medium (Recipe 1) or Stage 3B HPC medium (Recipe 2) with a ROCK inhibitor and 0.1% pluronic acid for 24 hours. Reactor wheel speed was set to 20 RPM for 3 hours and 25 RPM for 21 hours. The basal medium for both Stage 3 recipes is as follows: 50/50 IMDM/F12, 0.25% albumax, 100 g/ml vitamin c phosphate, 10 μg/ml insulin, 10 μg/ml holo-transferrin, 10 μM trolox, 4 ng/ml linoleic acid, 10 μM ethanolamine, 200 ng/ml cholesterol sulfate, 150 μM 1-thioglycerol, 10 μM oleic acid, 5 ng/ml sodium selenite, and 1% Penicillin-Streptomycin. Stage 3A HPC medium (Recipe 1) was made with the aforementioned basal medium and 20 ng/ml VEGF, 10 ng/ml FGF2, 20 ng/ml TPO, 50 ng/ml SCF, 20 ng/ml FLT3L. Stage 3B HPC medium (Recipe 2) was made with the aforementioned basal medium and 20 ng/ml VEGF, 10 ng/ml FGF2, 10 ng/ml IL-3, 15 ng/ml SCF, 20 ng/ml FLT3L, 130 μM lipoic acid, 4.5 μM SB434512, 25 nM GSK1016790A, 40 nM sphingosine-1-phosphate, and 30 ng/ml IGF-1.

On day 6, single cells were harvested by differential centrifugation. Aggregates and single cells were collected and centrifuged at 450 RPM for 1 second. The single cell suspension was collected, and aggregates were added back to the reactor with fresh Stage 3 media at 0.18M-0.3M cells/ml but without a ROCK inhibitor or pluronic acid. Single cells were cryopreserved in 10% DMSO and 90% knock out serum replacement. The same differential centrifugation and cryopreservation method was used daily on days 7-10.

Cryopreserved cells from days 6 to 10 were thawed, and viability, cell count, and HPC characterization were evaluated. HPCs were characterized by flow cytometry on markers CD43, CD34, CD38, CD45RA, CD90, CD33, CD71, and CD235. Further, qPCR was used to characterize the HPCs with markers HOXA9, SPINK2, MLLT3, HLF, RUNX1, CD45, and MECOM.

### Monocyte Differentiation

Stage 1-Day 0-8: Thawed HPCs were plated at 0.15M cells/ml in 15 ng/ml IL-3, 5 ng/ml M-CSF, 1% penicillin-streptomycin, and 1% GlutaMAX™ in Lonza X-Vivo 15. Fresh media was replaced after 4 days. Cells were tested by flow cytometry for characterization and tested for the expression of CD14, CD16, CD33, and CD45.

### Neutrophil Differentiation

Stage 1-Day 0-7: Thawed HPCs were plated at 0.15M cells/ml in 50 ng/ml GM-CSF, 20 ng/ml IL-6, 20 ng/ml IL-3, and 1% penicillin-streptomycin in StemSpan™ SFEM II. Fresh media was replaced after 3-4 days.

Stage 2-Day 7-14: 0.15M-1M cells/ml were moved into Stage 2 media in 2 μM AM580, 50 ng/ml G-CSF, and 1% penicillin-streptomycin in StemSpan™ SFEM II. Fresh media was added after 3-4 days. Cells were tested by flow cytometry for characterization and tested for the expression of CD11b, CD16, CD14, CD45, and CD66b.

### Erythroid Differentiation

Stage 1-Day 0-7: Thawed HPCs were plated at 80,000 cells/ml in erythroid differentiation medium consisting of 100 ng/ml SCF, 4 U/ml EPO, and 5 ng/ml IL-3 in either StemSpan™ SFEM II or the basal medium described above, which consists of 50/50 IMDM/F12, 0.25% albumax, 100 μg/ml vitamin c phosphate, 10 μg/ml insulin, 210 μg/ml holo-transferrin, 10 μM trolox, 4 ng/ml linoleic acid, 10 μM ethanolamine, 200 ng/ml cholesterol sulfate, 150 μM 1-thioglycerol, 10 μM oleic acid, 5 ng/ml sodium selenite, 1% penicillin-streptomycin, 0.5% fatty free acid BSA, 0.5% BSA, 200 nM LPA, 20 μM L-α-Phosphatidylcholine, and 1× Millipore sigma lipid mixture 1. Fresh media was replaced after 3-4 days.

Stage 2-Day 7-14: 0.5M cells/ml Stage 1 cells were moved into Stage 2 erythroid differentiation medium consisting of 100 ng/ml SCF and 4 U/ml EPO in either StemSpan™ SFEM II or trailhead basal medium, which consists of 50/50 IMDM/F12, 0.25% albumax, 100 μg/ml vitamin c phosphate, 10 μg/ml insulin, 210 μg/ml holo-transferrin, 10 μM trolox, 4 ng/ml linoleic acid, 10 μM ethanolamine, 200 ng/ml cholesterol sulfate, 150 μM 1-thioglycerol, 10 μM oleic acid, 5 ng/ml sodium selenite, 1% penicillin-streptomycin, 0.5% fatty free acid BSA, 0.5% BSA, 200 nM LPA, 20 μM L-α-Phosphatidylcholine, and 1× Millipore sigma lipid mixture 1. Fresh media was added after 3-4 days. Cells were tested by flow cytometry for characterization and tested for the expression of CD45, CD71, CD33, CD41, and CD235a.

### T-cell Differentiation

T-cell differentiation was conducted with thawed HPCs using the StemSpan™ T Cell Generation Kit (Cat #09940) and the protocol provided in Generation of T Cells Using STEMdiff™ or StemSpan™ T Cell Kits from Stemcell™ Technologies.

### Results

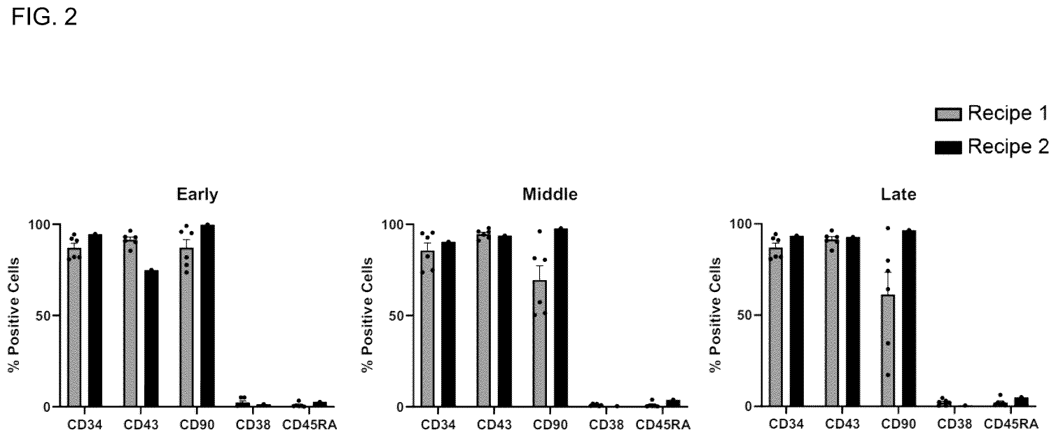

Flow Cytometry: the levels of CD34, CD43, CD90, CD38, and CD45RA expression were assessed in early, middle, and late HPCs. All showed high levels of CD34, CD43, and CD90 expression (FIG. 2).

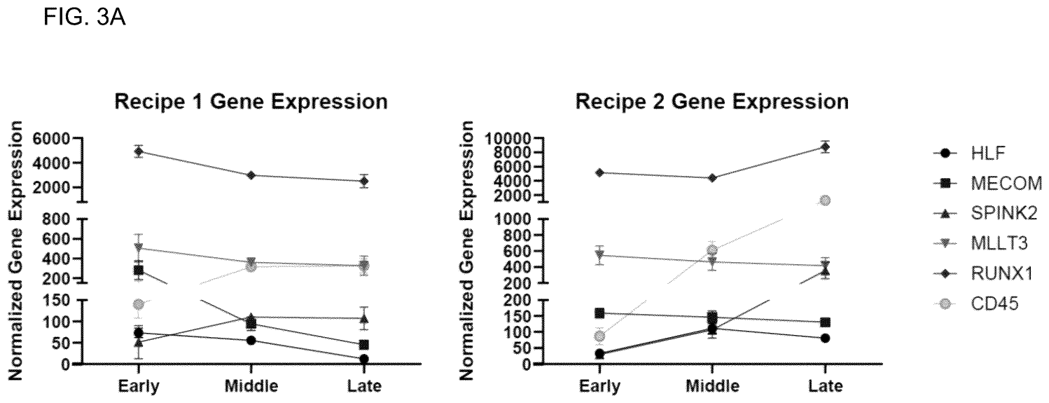

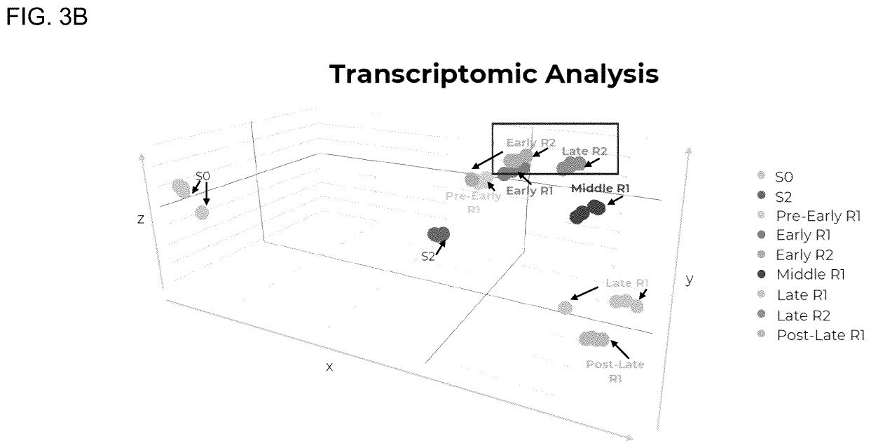

qPCR: the levels of HLF, MECOM, SPINK2, MLLT3, RUNX1, and CD45 gene expression were assessed in early, middle, and late HPCs. All were observed in the HPCs (FIG. 3A).

Bulk RNAseq analysis: the expression of HPC genes was dynamic and rapidly changed over time (FIG. 3B). HPCs cultured in Recipe 1 media progressed rapidly in Stage 3 media, while cells cultured in Recipe 2 maintained an earlier phenotype for a longer time.

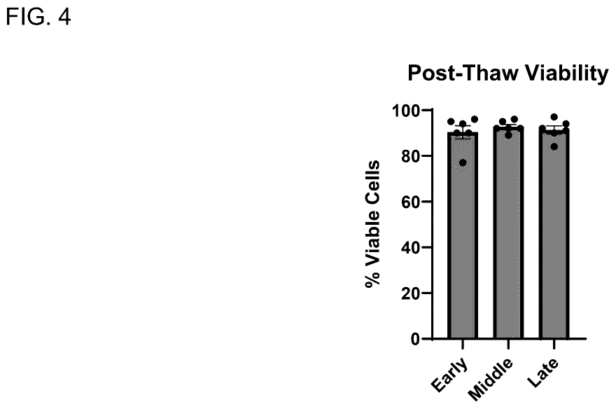

Viability: more than 90% of the HPCs were viable post-cryopreservation (FIG. 4).

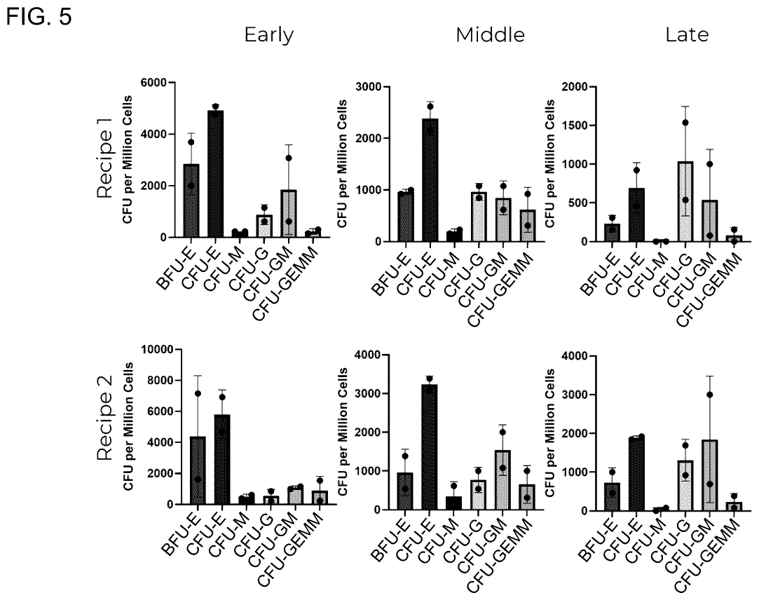

Colony-forming assay: early and middle HPCs had colonies of all lineages, while late cells lacked CFU-M colonies (FIG. 5).

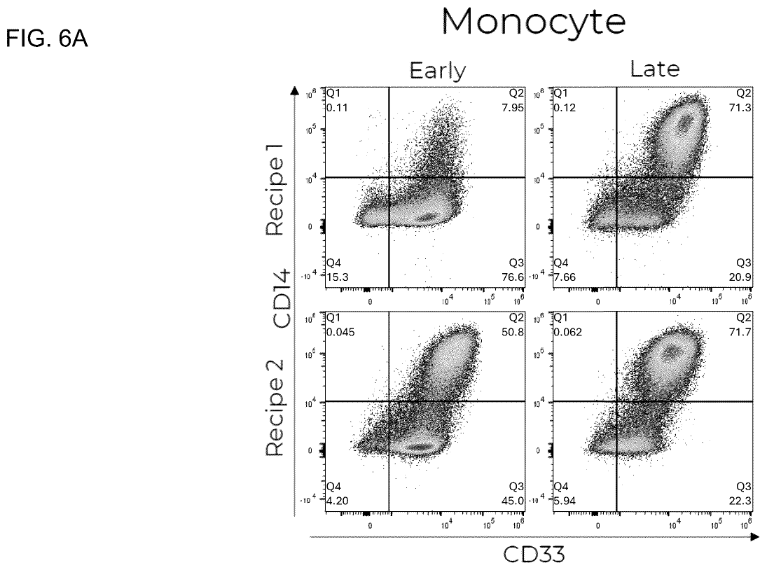

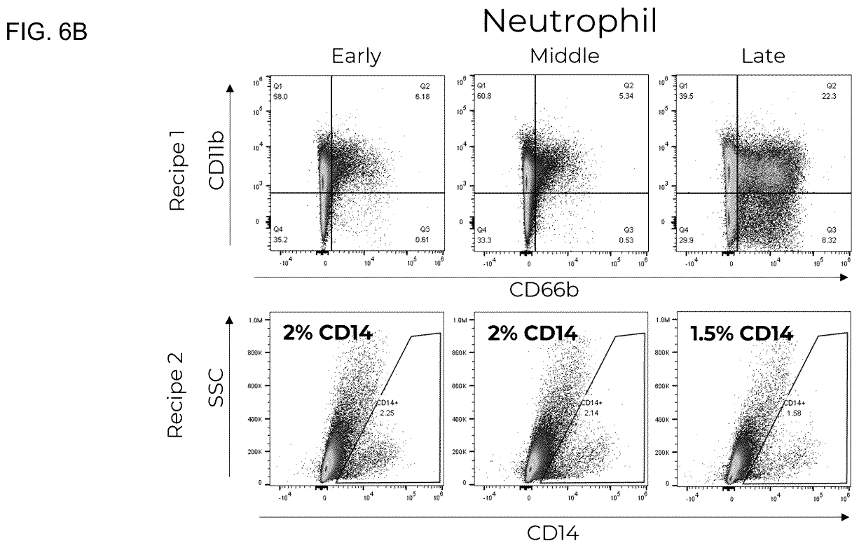

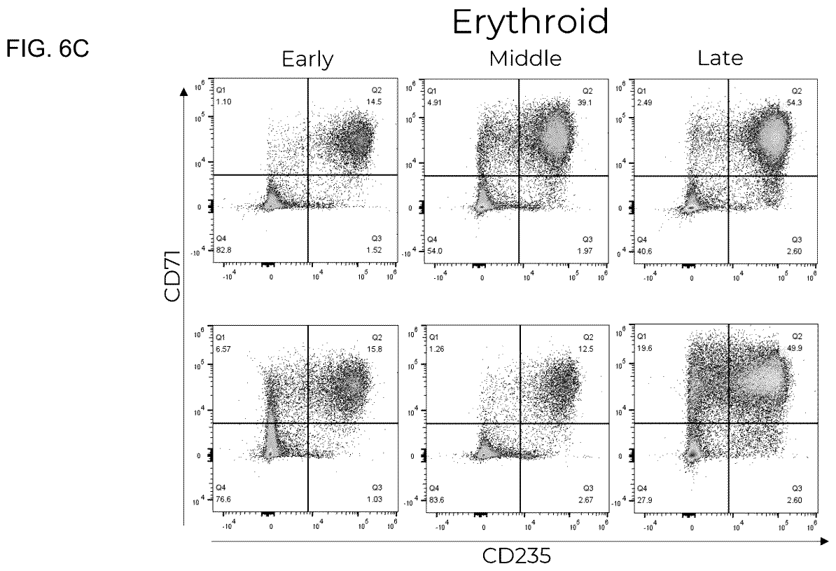

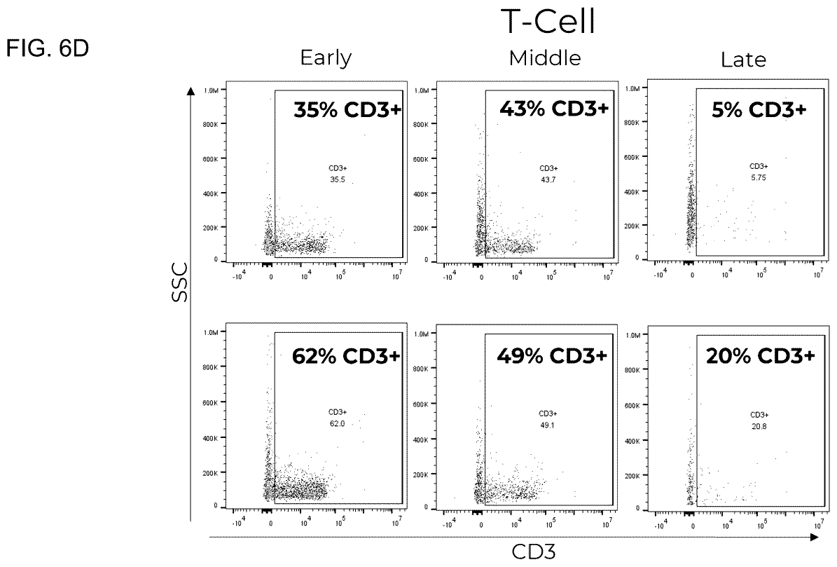

Flow Cytometry: HPCs differentiated into monocytic, erythroid, neutrophil, and lymphoid lineages using the protocols described herein (FIGS. 6A-6D).

### Conclusion

These findings demonstrate that HD-DoE is an effective tool for developing a scalable differentiation protocol for generating a large number of iPSC-derived HPCs with high viability and CD34 expression. HPC gene expression profile and the robust multi-lineage potential found in vitro validate the protocol's efficiency. These results suggest the potential of this method for producing donor-independent HPCs for research applications and disease modeling, highlighting an unbiased strategy for scalable blood cell production.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims. Other embodiments are within the claims.

1. A method of generating a population of human CD34+, CD43+, and CD90+ hematopoietic progenitor cells (HPCs), the method comprising culturing a population of human CD34+ hematopoietic stem cells (HSCs) in a culture medium comprising a vascular endothelial growth factor receptor (VEGFR) agonist, a fibroblast growth factor receptor (FGFR) agonist, a c-kit agonist, a thrombopoietin receptor (TPOR) agonist, and an fms-like tyrosine kinase 3 (FLT3) agonist for at least 12 hours.

2. The method of claim 1, wherein:
(a) the VEGFR agonist is selected from the group consisting of VEGF, Gremlin, PG-007, Taurocholic acid, KLTWQELYQLKYKGI, and a peptide derived from VEGF;
(b) the FGFR agonist is selected from the group consisting of FGF2, SUN11602, FGF1, FGF3, FGF4, FGF5, FGF6, FGF8, FGF10, FGF17, FGF19, FGF20, FGF21, FGF22, and FGF23;
(c) the TPOR agonist is selected from the group consisting of thrombopoietin (TPO), eltrombopag, TA-316, TPO agonist 1, avatrombopag, and lusutrombopag; and/or
(d) the FLT3 agonist is selected from the group consisting of FLT3L, CDX-301, GS-3583, FLT3L-Fc, and a peptide derived from FLT3L.

3. The method of claim 1, wherein:
(a) the VEGFR agonist is VEGF;
(b) the FGFR agonist is FGF2;
(c) the c-kit agonist is stem cell factor (SCF);
(d) the TPOR agonist is TPO; and/or
(e) the FLT3 agonist is FLT3L.

4. The method of claim 3, wherein:
(a) VEGF is present in the culture medium at a concentration of 1-400 ng/ml;
(b) FGF2 is present in the culture medium at a concentration of 2-150 ng/ml;
(c) SCF is present in the culture medium at a concentration of 2-200 ng/ml;
(d) TPO is present in the culture medium at a concentration of 1-200 ng/ml; and/or
(e) FLT3L is present in the culture medium at a concentration of 0.2-200 ng/ml.

5-15. (canceled)

16. A method of generating a population of human CD34+, CD43+, and CD90+ HPCs, the method comprising culturing a population of human CD34+ HSCs in a culture medium comprising a VEGFR agonist, an FGFR agonist, a c-kit agonist, an interleukin 3 receptor (IL-3R) agonist, an antioxidant, a FLT3 agonist, a transforming growth factor beta (TGF-β) antagonist, a transient receptor potential vanilloid 4 (TRPV4) agonist, a bioactive phospholipid, and an insulin-like growth factor 1 (IGF-1) receptor agonist for at least 12 hours.

17. The method of claim 16, wherein:
(a) the VEGFR agonist is selected from the group consisting of VEGF, Gremlin, PG-007, Taurocholic acid, KLTWQELYQLKYKGI, and a peptide derived from VEGF;
(b) the FGFR agonist is selected from the group consisting of FGF2, SUN11602, FGF1, FGF3, FGF4, FGF5, FGF6, FGF8, FGF10, FGF17, FGF19, FGF20, FGF21, FGF22, and FGF23;
(c) the IL-3R agonist is selected from the group consisting of IL-3, SC-65461, SC-55494 (Synthokine 1), and a peptide derived from IL-3;
(d) the antioxidant is selected from the group consisting of lipoic acid, N-acetyl-L-Cysteine, ascorbic acid, sodium ascorbate, glutathione, ebselen, α-tocopherol, β-tocopherol, δ-tocopherol, γ-tocopherol, uric acid, and ubiquinol;
(e) the FLT3 agonist is selected from the group consisting of FLT3L, CDX-301, GS-3583, FLT3L-Fc, and a FLT3L peptide;
(f) the TGF-β antagonist is selected from the group consisting of SB431542, A 83-01, GW788388, SB525334, SB505124, TP0427736, RepSox, SD-208, Galunisertib, IN-1130, LY2109761, LY550410, and TEW-7197;
(g) the TRPV4 agonist is selected from the group consisting of GSK1016790A, 4α-Phorbol 12,13-didecanoate, N-Arachidonoyl Taurine, and RN-1747;
(h) the bioactive phospholipid is selected from the group consisting of sphingosine-1-phosphate (S1P), lysophosphatidic acid (LPA), ceramide-1-phosphate (C1P), and lysophosphatidylcholine (LPC); and/or
(i) the IGF-1 receptor agonist is selected from the group consisting of IGF-1, IGF-1-Ado, X10, Mecasermin, IGF-2, Insulin, Rg5, IGF-1 24-41, IGF-1 30-41, des(1-3)IGF-1, IGF-1 LR3, and Demethylasterriquinone B1.

18. The method of claim 17, wherein:
(a) the VEGFR agonist is VEGF;
(b) the FGFR agonist is FGF2;
(c) the c-kit agonist is stem cell factor (SCF);
(d) the IL-3R agonist is IL-3;
(e) the antioxidant is lipoic acid;
(f) the FLT3 agonist is FLT3L;
(g) the TGF-β antagonist is SB431542;
(h) the TRPV4 agonist is GSK1016790A;
(i) the bioactive phospholipid is S1P; and/or
(i) the IGF-1 receptor agonist is IGF-1.

19. The method of claim 18 wherein:
(a) VEGF is present in the culture medium at a concentration of 1-400 ng/ml;
(b) FGF2 is present in the culture medium at a concentration of 2-150 ng/ml;
(c) SCF is present in the culture medium at a concentration of 2-200 ng/ml;
(d) IL-3 is present in the culture medium at a concentration of 0.02-100 ng/ml;
(e) lipoic acid is present in the culture medium at a concentration of 1-260 μM;
(f) FLT3L is present in the culture medium at a concentration of 0.2-200 ng/ml;
(g) SB431542 is present in the culture medium at a concentration of 0.2-50 μM;
(h) GSK1016790A is present in the culture medium at a concentration of 0.2-500 nM;
(i) S1P is present in the culture medium at a concentration of 0.005-1 μM;
(j) IGF-1 is present in the culture medium at a concentration of 0.2-200 ng/ml.

20-45. (canceled)

46. An isolated cell culture comprising at least 100 million human HPCs in a culture medium comprising:
(a) a VEGFR agonist, an FGFR agonist, a c-kit agonist, a TPOR agonist, and a FLT3 agonist; or
(b) a VEGFR agonist, an FGFR agonist, a c-kit agonist, an IL-3R agonist, an antioxidant, a FLT3 agonist, a TGF-β antagonist, a TRPV4 agonist, a bioactive phospholipid, and an IGF-1 receptor agonist.

47. The isolated cell culture of claim 46, wherein the HPCs are CD34+, CD43+, and CD90+.

48. The isolated cell culture of claim 46, wherein:
a. after 42-54 hours of culture, the HPCs express normalized gene levels of HLF in a range of 35-140, MECOM in a range of 140-560, SPINK2 in a range of 25-100, CD45 in a range of 43-172 and MLLT3 in a range of 250-1000;
b. after 66-78 hours of culture, the HPCs express normalized gene levels of HLF in a range of 25-110, MECOM in a range of 47-188, SPINK2 in a range of 50-200, CD45 in a range of 158-632 and MLLT3 in a range of 180-720; and
c. after 90-102 hours of culture, the HPCs express normalized gene levels of 5HLF in a range of 6-24, MECOM in a range of 22-90, SPINK2 in a range of 50-100, CD45 in a range of 162-648 and MLLT3 in a range of 163-652.

49. The isolated cell culture of claim 46, wherein the HPCs possess, per million cells plated, in a colony-forming unit assay:
a. after 42-54 hours of culture, 1000-7000 burst forming units for erythroid potential; 4000-7000 colony-forming units for erythroid potential; 0-1000 colony-forming units for macrophage potential; 0-3000 colony-forming units for granulocyte potential; 500-2500 colony-forming units for macrophage-granulocyte potential; and 0-2000 colony-forming units for granulocyte-erythroid-macrophage-megakaryocyte potential;
b. after 66-78 hours of culture, 500-2000 burst forming units for erythroid potential; 4000-6000 colony-forming units for erythroid potential; 0-1000 colony-forming units for macrophage potential; 0-3000 colony-forming units for granulocyte potential; 0-4000 colony-forming units for macrophage-granulocyte potential; and 0-2000 colony-forming units for granulocyte-erythroid-macrophage-megakaryocyte potential; and
c. after 90-102 hours of culture, 500-1000 burst forming units for erythroid potential; 1000-4000 colony-forming units for erythroid potential; 0-100 colony-forming units for macrophage potential; 0-3000 colony-forming units for granulocyte potential; 0-4000 colony-forming units for macrophage-granulocyte potential; and 0-1000 colony-forming units for granulocyte-erythroid-macrophage-megakaryocyte potential.

50. The isolated cell culture of claim 46, wherein the HPCs are differentiated from induced pluripotent stem cells (iPSCs).

51. The isolated cell culture of claim 46, wherein the HPCs are derived from umbilical cord blood.

52. The isolated cell culture of claim 46, wherein the HPCs are derived from bone marrow.

53. The isolated cell culture of claim 46, wherein the HPCs are genetically engineered.

54-55. (canceled)

56. The isolated cell culture of claim 46, wherein:
a. after 42-54 hours of culture, the HPCs express normalized gene levels of HLF in a range of 15-66, MECOM in a range of 80-316, SPINK2 in a range of 15-60, CD45 in a range of 43-172 and MLLT3 in a range of 274-1096;
b. after 66-78 hours of culture, the HPCs express normalized gene levels of HLF in a range of 55-220, MECOM in a range of 73-292, SPINK2 in a range of 50-200, CD45 in a range of 305-1200 and MLLT3 in a range of 223-930; and
c. after 90-102 hours of culture, the HPCs express normalized gene levels of HLF in a range of 40-160, MECOM in a range of 65-262, SPINK2 in a range of 178-712, CD45 in a range of 625-2500 and MLLT3 in a range of 208-834.

57. The isolated cell culture of claim 46, wherein the HPCs possess, per million cells plated, in a colony-forming unit assay:
a. after 42-54 hours of culture, 1500-7000 burst forming units for erythroid potential; 4000-7000 colony-forming units for erythroid potential; 0-1000 colony-forming units for macrophage potential; and 0-3000 colony-forming units for granulocyte potential; 500-2500 colony-forming units for macrophage-granulocyte potential; and 0-2000 colony-forming units for granulocyte-erythroid-macrophage-megakaryocyte potential;
b. after 66-78 hours of culture, 500-2000 burst forming units for erythroid potential; 4000-6000 colony-forming units for erythroid potential; 0-1000 colony-forming units for macrophage potential; 0-3000 colony-forming units for granulocyte potential; 0-4000 colony-forming units for macrophage-granulocyte potential; and 0-2000 colony-forming units for granulocyte-erythroid-macrophage-megakaryocyte potential; and
c. after 90-102 hours of culture, 500-1000 burst forming units for erythroid potential; 1000-4000 colony-forming units for erythroid potential; 0-100 colony-forming units for macrophage potential; and 0-3000 colony-forming units for granulocyte potential; 0-4000 colony-forming units for macrophage-granulocyte potential; and 0-1000 colony-forming units for granulocyte-erythroid-macrophage-megakaryocyte potential.

58-61. (canceled)
